(19) United States
(12) Patent Application Publication
(10) Pub. No.: US 2021/0068646 A1
(43) Pub. Date: Mar. 11, 2021

(54) PORTABLE OTOSCOPE

(71) Applicant: REMMIE, INC., Wilmington, DE (US)

(72) Inventors: Jane Yuqian ZHANG, Bothell, WA (US); Yuk Kwan LEE, Bothell, WA (US); Yifu ZHANG, Bothell, WA (US)

(21) Appl. No.: 17/044,285

(22) PCT Filed: Apr. 2, 2019
(86) PCT No.: PCT/US2019/025437

§ 371 Date: Sep. 30, 2020

Related U.S. Application Data
(60) Provisional application No. 62/651,626, filed on Apr. 2, 2018.

(51) Int. Cl. A61B 1/227; A61B 1/04; A61B 1/32; A61B 1/07; A61B 1/00; A61B 5/00

(52) U.S. Cl. CPC A61B 1/227; A61B 1/04; A61B 1/32; A61B 2576/02; A61B 1/00009; A61B 1/00016; A61B 5/7267; A61B 1/07

(57) ABSTRACT

An otoscope configured to be used together with a host device, such as a smart phone or other handheld mobile devices. The otoscope can utilize features on the hand-held mobile device, such as camera, software, and display, to allow a user view a person's ear canal and tympanic membrane or eardrum. In some embodiments, the otoscope may comprise a housing; a light source; an speculum; an optical redirection component; one or more image sensors; image-processing circuitry; and communication circuitry configured to transmit the sensed image to the host device.

### CROSS-REFERENCE TO RELATED APPLICATIONS

This application claims benefit of U.S. Provisional Patent Application No. 62/651,626, filed Apr. 2, 2018, which is hereby incorporated by reference in its entirety.

### FIELD

This disclosure relates generally to otoscope and, more particularly, to the design and use of an otoscope configured to be used together with a hand-held mobile device.

### BACKGROUND

An otoscope is a device used to look into the ears and nasopharynx. Health care providers use otoscopes to screen for illness during regular check-ups and also to diagnose ear, nasal, or other symptoms; similarly, otoscopes can be used in other fields, such as veterinarian diagnosis and care. An otoscope allows visual inspection of the ear canal, the tympanic membrane, the eardrum, or the nasopharynx. Many otoscopes consist of a handle and a head. The head contains a light source and a magnifying lens. The front end of the otoscope often has an attachment for a speculum. Many otoscopes used in doctors' offices are wall-mounted while others are portable. Wall-mounted otoscopes are attached by a flexible power cord to a base, which holds the otoscope when it's not in use and also serves as a source of electric power. Portable models are powered by batteries in the handle.

### SUMMARY

Examples of the disclosure are directed to an otoscope configured to be used together with a host device, such as a smart phone, other handheld mobile devices, or other suitable host device. In some embodiments, the otoscope utilizes features in the host device, such as a processor, software, and display, to allow a user view a person's ear canal and tympanic membrane, eardrum, and nasopharynx. In some embodiments, the otoscope comprises a housing. In some embodiments, the otoscope comprises a light source. In some embodiments, the otoscope comprises a speculum configured to be attached to the housing and configured to pass light reflected from the light source first through a first opening of the speculum (i.e., the distal opening), then through a second opening of the speculum (i.e., the proximal opening). In some embodiments, the otoscope comprises one or more image sensors to sense an image formed by light returned from the examined area. In some embodiments, the otoscope comprises an optical redirection component, such as a lens, in the housing and configured to direct returned light to the one or more image sensors. In some embodiments, the light source is configured to direct light out of the speculum without the use of mirrors or lenses. In some embodiments, the otoscope comprises communication circuitry configured to transmit the sensed image to the host device. In some embodiments, the otoscope transmits the sensed image is manipulated and/or analyzed by a processor, and software executed by the processor is configured to produce one or more features such as colorimetric readout for dual-imaging-spectral capture, quality factor readout, and/or likelihood of infection. In some embodiments, the analysis is performed based on machine-learning algorithms (e.g., embedded within the software). By utilizing existing features available in the host device, the various embodiments can allow the user view and record images of an examinee's ear canal and ear drum. In some embodiments, the housing of the otoscope includes one or more processors and performs some or all of the analysis described above.

### BRIEF DESCRIPTION OF THE DRAWINGS

FIG. 1 illustrates a perspective view an exemplary otoscope.

FIG. 2 illustrates a perspective sectional view of a portion of an exemplary otoscope.

FIG. 3 illustrates a cross-sectional view of an exemplary otoscope.

FIG. 4 illustrates an exterior view of an exemplary otoscope.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

Currently, otoscopes can be either wall-mounted devices or portable devices. Wall-mounted otoscopes are attached by a flexible power cord to a base. The base holds the otoscope when it's not in use and also serves as a source of electric power. Portable models are powered by batteries in the handle. Wall mounted otoscopes are generally not intended for household uses due to its permanent nature. Portable otoscopes are often inaccessible for household use due to the high cost resulting from complex and expensive components. However, an ear infection often requires regular visual inspections to monitor and treat, thus requiring multiple office visits to a medical professional. Thus, it is desired to have an otoscope for household use that is capable of easily capturing images of a patient's ear and transmitting the images to a medical professional without requiring the patient to visit the medical professional.

In some embodiments, a portable otoscope is a mobile-handheld device and utilizes features such as a camera, a light source, an image processor and communication circuitry capable of transmitting one or more images to a host device. In some embodiments, the portable otoscope can be shaped like a pen or can have any other shape. In some embodiments, a host device includes a processor to analyze the images received from the otoscope and a display to display the received images. It is understood that although the disclosure herein describes the examination and imaging of an otoscope for viewing a patient's ears, the disclosure is not limited to only ears (e.g., ear canal, ear drum) and can also be applied to noses (nasopharynx) or other suitable body cavity.

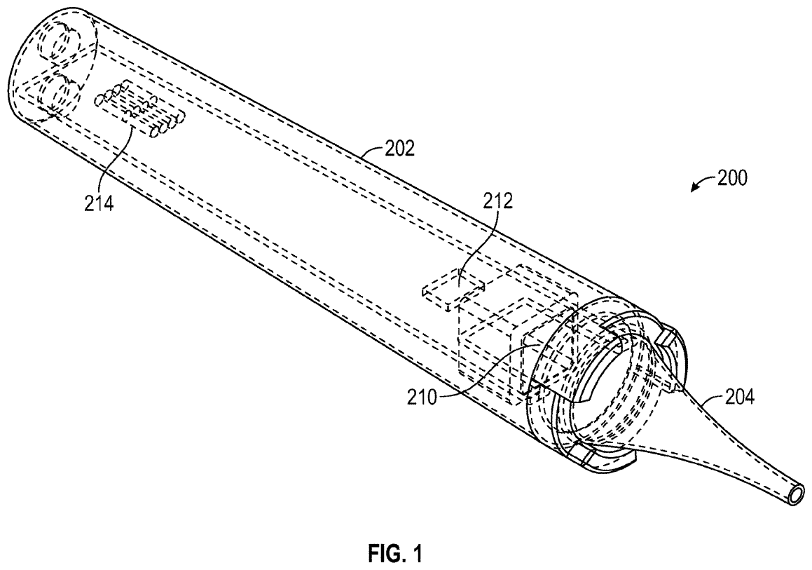

FIG. 1 illustrates a perspective view of an exemplary otoscope 200. In some embodiments, otoscope 200 includes a housing 202 that defines the shape and size of otoscope 200. In some embodiments, housing 202 is cylindrical. In some embodiments, housing 202 is curved, includes a handle portion and/or otherwise has an ergonomic shape. In some embodiments, housing 202 can be composed of plastic or metal. In some embodiments, housing 202 can be manufactured using injection molding, Computer Numerical Control (CNC) subtractive machining, or computerized additive manufacturing (e.g., 3-D printing). In some embodiments, housing 202 can be any suitable shape or size.

In some embodiments, speculum 204 is attached to one end of housing 202. In some embodiments, speculum 204 allows a view inside a patient's ear canal. For example, in some embodiments speculum 204 is a hollow funnel shape and is configured to be placed in or near a patient's ear canal, thus ensuring a clear visual path through the interior of speculum 204 and into the patient's ear canal. In some embodiments, the speculum is designed to pass light between the patient's ear canal and the otoscope. For example, light from a light source in the otoscope can pass through the speculum into the ear canal and light that is reflected off of surfaces in the ear canal can pass from the ear canal back through the speculum and into the otoscope. In some embodiments, the speculum 204 has a larger opening (e.g., distal opening) that is attached to housing 202 and a smaller opening (e.g., proximal opening) that placed in or near the patient's ear canal.

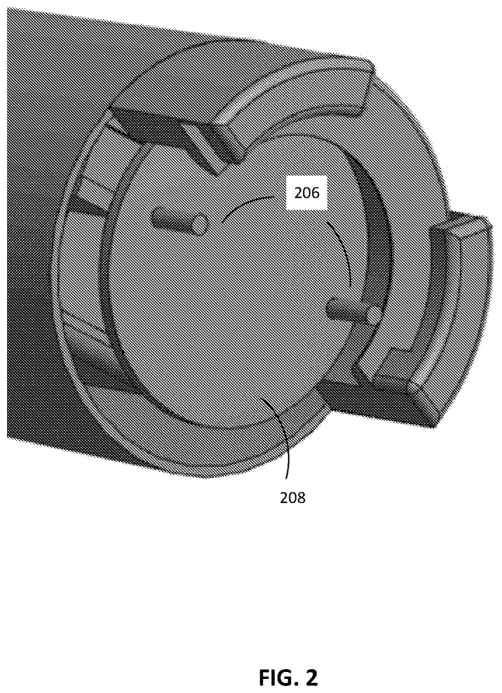

In some embodiments, the speculum 204 can be disposable, meaning that the speculum 204 can be removed from housing 202 without the use of any tools and without disassembling housing 202. In some embodiments, a disposable speculum allows for quick disposal of used speculums and installation of a fresh, sterile speculum, thus minimizing cross-contamination between uses and/or users. In some embodiments, the speculum is a rubber or other suitable polymer material. In some embodiments, the speculum is washable and/or reusable. In some embodiments, housing 202 includes one or more attachment mechanisms via which speculum 204 is attached to housing 202, as described in more detail below with respect to FIG. 2.

In some embodiments, otoscope 200 includes one or more light sources mounted inside or on housing 202, as described in more detail below with respect to FIG. 2. In some embodiments, the one or more light sources are mounted such that light from the light sources are directed through the speculum and into the patient. In some embodiments, the light sources can be any one of LED, incandescent bulbs, or any other suitable light source.

In some embodiments, otoscope 200 includes one or more image sensors 210. In some embodiments, image sensors 210 can be digital or analog image sensors and the resulting images can be digital or analog. In some embodiments, image sensors 210 can be an array of image sensors, such as a charge coupled device (CCD) grid, a complementary metal oxide semiconductor (CMOS) sensor, or any other suitable image sensor. In some embodiments, the one or more image sensors 210 are mounted inside of housing 202 and is configured to receive light through speculum 204 directly into image sensors 210 (e.g., the image sensors share the same longitudinal axis as speculum 204). In some embodiments, one or more mirrors or lenses can reflect light into image sensors 210 (e.g., if image sensors 210 are not co-axial with speculum 204).

In some embodiments, speculum 204 is substantially centered on housing 202 and image sensors 210 are substantially centered in housing 202 such that the center of the view through the speculum 204 aligns with the center of the viewable area of the one or more image sensors 210. In some embodiments, aligning speculum 204 with image sensors 202 increases the field of view and improves the quality of the image captured by the image sensors 210. In some embodiments, the larger opening of speculum 204 and smaller opening of speculum 204 are centered (e.g., longitudinally aligned) with the one or more image sensors 210. Various shapes, sizes, and materials are suitable for creating the housing 202 are contemplated.

In some embodiments, the one or more image sensors 210 receive (e.g., sense, detect, etc.) an image of the user's ear when light from one or more light sources (e.g., from otoscope 200 or from an external light source) is reflected from a user's ear, travels through speculum 204, and is detected by the one or more image sensors 210. In some embodiments, the sensors can extract color and intensity from the received light. In some embodiments, data from the one or more sensors or pixels of image sensor 210 is aggregated to form a digital image corresponding to the imaged location (e.g., a patient's ear canal). In some embodiments, the digital image can be either still (e.g., still image, photograph, etc.) or continuous (e.g., video or clip, etc.). In some embodiments, one or more continuous images (e.g., periodically sampled) captured in real-time can comprise a digital video (e.g., wherein the digital video comprises a plurality of frames of still images). Various implementations of the one or more digital image sensors are contemplated.

FIG. 2 illustrates a perspective sectional view of a portion of an exemplary otoscope 200. In some embodiments, FIG. 2 illustrates the end of housing 202 with the speculum removed for illustrative purposes. In some embodiments, as described above, otoscope 200 includes one or more light sources and one or more attachment mechanisms. As shown in FIG. 2, in some embodiments, otoscope 200 includes two light emitting diodes (LEDs) 206. In some embodiments, the light emitting diodes 206 are mounted on the end of housing 202 of otoscope 200. In some embodiments, the light emitting diodes 206 are placed across from the center of housing 202. In some embodiments, placing light emitting diodes 206 on the sides of the center of housing 202 allows reflected light to enter housing 202 through the center of housing 202 while still allowing light to be emitted out of the speculum. In some embodiments, more or fewer light sources can be used. In some embodiments, the light sources can be placed in any pattern that allows light to be transmitted out through the speculum.

In some embodiments, the size, placement and shape of the light sources is configured to increase the amount of light passing through the speculum 204. For example, in some embodiments, the light source 206 is placed between the speculum 204 and the optical redirection component 208 substantially along the same direction. In some embodiments, light source 206 is placed out of the field of view of the one or more digital image sensors. In some embodiments, light source 206 is not placed between the speculum 204 and the optical redirection component 208, and a flexible optical guide, such as an optical fiber, is used to pass light from the light source 206 through the speculum. In some embodiments, the placement of the light sources 206 reduces or minimizes obstruction to the field of view of the one or more image sensors 210. In some embodiments, optical redirection component 208 is a lens or mirror that reflects light from the one or more light sources toward the speculum. In some embodiments, optical redirection component 208 aligns light such that the light that travels through the speculum is substantially one-directional (e.g., as opposed to scattered). In some embodiments, the length and shape of the speculum ensures that light that travels through the speculum is substantially one-directional. In some embodiments, optical redirection component 208 includes a hole such that light that travels inwards through the speculum can enter housing 202 and not reflected, scattered, or otherwise lost. In some embodiments, optical redirection component 208 redirects light that enters speculum 204 into the one or more image sensors 210.

As discussed above, in some embodiments, housing 202 includes one or more attachment mechanisms for attaching the speculum to housing 202. In some embodiments, housing 202 has one or more ledges on the end to which the speculum 204 attaches. In some embodiments, the ledges are shaped to create a recess under each ledge. In some embodiments, the speculum 204 has one or more lips protruding from the end that complements the ledges on housing 202. In some embodiments, the protruding lips of speculum 204 attaches to the housing 202 via fitting underneath the ledges (e.g., in the recess). In some embodiments, to attach the speculum 204 to the housing 202, a user aligns the lips on the speculum 204 with sections of housing 202 that do not have ledges and twists the speculum into a position such that the lips on speculum 204 fit within the recesses under the ledges.

In some embodiments, speculum 204 can be attached to housing 202 by threaded fastening. In such exemplary embodiments, the speculum 204 may be designed to fit within a portion of internally-threaded inner surface of the housing 202 and include external helical threads that tighten with the internal threads on the housing 202 by twisting. In another embodiment, the speculum 204 and the housing 202 may be connected using a snap-fit joint such as a cantilever or an annular snap-fit. In an exemplary embodiment with cantilever snap-fit, speculum 204 may include one or more cantilevers each having a protruding part (e.g., a hook), and the housing 202 may include a corresponding number of undercuts). During the connection, the cantilevers can be briefly deflected, and each protruding parts can fit inside and catch an undercut, thereby attaching the speculum 204 and housing 202. In some embodiments, speculum 204 is a flexible polymer material and attachment is achieved by fitting the speculum around or onto a piece of housing 202 such that the elastic properties of the speculum hold the speculum in place. Various methods of attaching the speculum 204 to the housing 202 are contemplated.

Returning to FIG. 1, in some embodiments, otoscope 200 further includes communication circuitry 212 electrically coupled to the one or more image sensors 210. In some embodiments, the communication circuitry is designed to transmit digital images sensed by the image sensors to a host device for further processing and display. In some embodiments, the host device is a hand-held mobile device, such as a smart phone. In some embodiments, the hand-held mobile device includes a host processor, such as a central processing unit (CPU), a graphical processing unit (GPU), or any other suitable processing device. In some embodiments, various applications (e.g., mobile apps) can access the received images and perform analysis or other type of processing on the analysis. In some embodiments, the host device includes a display to display the image received from otoscope 200 either after further processing by various software on the host device or without further processing by the software on the host device. In some embodiments, the communication circuitry transmits the sensed images to the hand-held mobile device through a wired connection. For example, a wired connection using Universal Serial Bus (USB) protocol or a proprietary protocol may be established between the otoscope 200 and the hand-held mobile device. In some preferred embodiments, the communication circuitry 212 transmits the sensed images wirelessly to the hand-held mobile device. For example, in some embodiments, the communication circuitry 212 establishes a wireless connection with the hand-held mobile device using a Bluetooth protocol. In other embodiments, the communication circuitry 212 establishes a connection to a Wi-Fi network router using a Wi-Fi protocol, and the host device may likewise establish a connection with the same Wi-Fi network router. In some embodiments, the communication circuitry 212 transmits the sensed images to the hand-held mobile device through the Wi-Fi router. In some embodiments, the communication circuitry 212 employs a wireless communication protocol that aggregates data into packets and transmits the packets according to the communication protocol, thus the communication circuitry 212 may further comprise a computer processor, such as a micro-controller, and computer-readable storage media, such as Random-Access Memory (RAM). Various wired or wireless implementations of the communication circuitry and the connection to the host device are contemplated. In some embodiments, communication circuitry 212 hosts (e.g., creates) its own wireless network (e.g., Wi-Fi Direct, ad-hoc wireless connection, etc.) and the host device establishes a connection directly with the otoscope-hosted wireless network and transmits data over the otoscope-hosted wireless network. In some embodiments, other wireless protocols can be used such as NFC, IR, RF, etc. In some embodiments, the otoscope may transmit the sensed image to be processed by software on the host processor.

In some embodiments, the otoscope 200 may be configured to transmit the sensed image to be processed by software on the host processor. For example, in some embodiments, the host processor executes a mobile application to display and analyze the image captured by the otoscope 200. Sometimes, the infected tissues can lead to change in the appearance of the infected tissues, such as the color of the tissue. For example, healthy eardrums appear clear and pinkish-gray, whereas infected eardrums can appear red and swollen due to fluid buildup behind the membrane. To aid diagnosis, in some embodiments, the software executed by the host processor may be configured to provide a colorimetric analysis of the captured image, in addition to displaying the captured image. In some embodiments, the software may be configured to determine a quality factor indicating the level of health of the viewed area. In some embodiments, the software may determine the likelihood that the area being examined is infected based on the physical features of the patient, the colorimetric analysis and/or the quality factor.

In some embodiment, the determination of quality factor and likelihood of infection can be determined by one or more machine-learning algorithms embedded within the software. For example, the software analyzes the various images to determine the existence of one or more characteristics (e.g., physiological characteristics) within the images (e.g., buildup, infection, redness, or any other abnormality). For example, the software analyzes the various images, identifies various features within the image (e.g., physical features of the patient, color, etc.) that are associated with known different quality factors, likelihood of buildup, and/or likelihood of infection and determines one or more statistical correlation between colorimetric data and quality factors or likelihood of infection. In some embodiments, during a training mode for a machine learning model, a plurality of training images that have been pre-identified as having or not having an ear infection are provided to the machine learning model (e.g., identified as having the one or more physiological characteristics). In some embodiments, the machine learning model identifies different features in the images and assigns different weights to the features. In some embodiments, the different weights represent the amount in which the corresponding feature is indicative of likelihood of buildup, likelihood of infection, etc. In some embodiments, the machine learning model improves over time with the number of training images used. In some embodiments, the machine learning model re-adjusts the weights and/or identifies more or fewer features to improve the accuracy of the predictions. In some embodiments, the machine improves over previous training sessions by combining analysis from new training images to the data from previous training sessions (e.g., the identified features and/or assigned weights).

In some embodiments, after training the machine learning model, an image is analyzed by the machine learning model to identify the appropriate features and apply the learned weights to the features. In some embodiments, after applying the weights to the features, the machine learning model predicts whether the imaged ear does or does not have an ear infection, fluid buildup, and/or any other abnormality or characteristic. In some embodiments, other methods of analyzing and determining whether the user has an ear infection are possible. In some embodiments, the software supports one or more patient profiles. In some embodiments, images can be associated with a patient profile such that the software can track and organize images for different patients. Various embodiments and implementations are contemplated and disclosed herein.

In some embodiments, the otoscope 200 may receive power from the host device. For example, if a wired connection exists between the otoscope 200 and the host device, the host device may send electrical power to the otoscope 200 through the wired connection in order to power the light source 206, the image sensors 210, the communication circuitry 212, and other various electronic components included in the otoscope. In other embodiments, the otoscope may also include a battery 214, which can receive electrical charge through the wired connection. In other embodiments, there may not be a wired connection between otoscope 200 and the hand-held mobile device, and battery 214, which may power the various electronic components included in the otoscope during the otoscope's operation, can be charged using other charging devices, and the charge in battery 214.

In some embodiments, otoscope 200 further includes electronic circuitry configured to process images from the one or more image sensors before transmitting the image to another device (e.g., host device). In some embodiments, the electronic circuitry can process images by, for example, performing one or more operations such as pre-processing, sampling, filtering, pro-processing, transforming, formatting, and storing the digital signals obtained from the image sensors. In some embodiments, the digital signal can be compressed into transmittable data format.

It is understood that although the analysis and processing steps are disclosed as being performed by software on a host device, any or all of the analysis and processing steps can be performed by the otoscope itself or by a cloud or server computing device. For example, the otoscope can include one or more processors and software modules to analyze and/or process the images. In some embodiments, the otoscope can include a display to display the image and/or analysis results. In some embodiments, the otoscope and the host device can divide the above-described tasks. In some embodiments, the otoscope or host device can upload the images to a server to perform the analysis and processing steps and receive the results for display.

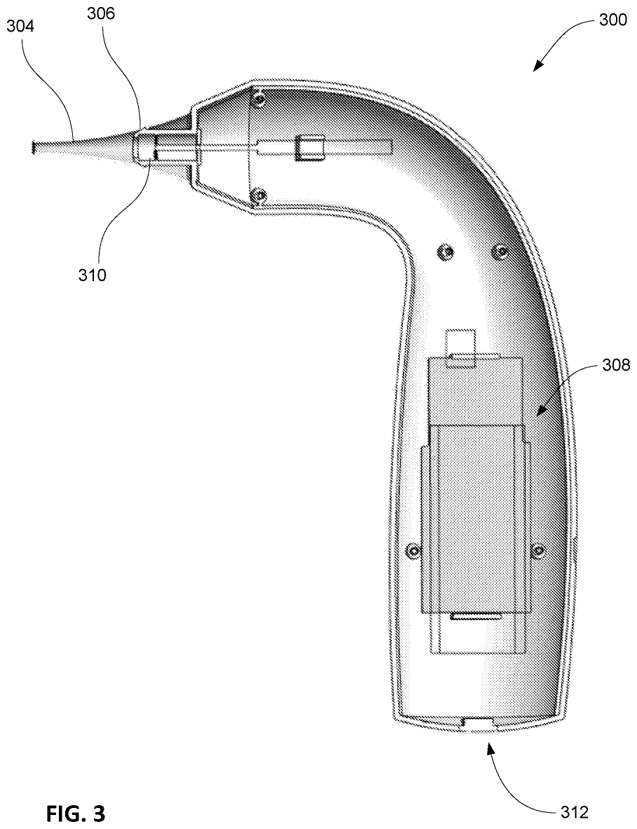

FIG. 3 illustrates a cross-sectional view of an exemplary otoscope 300. In some embodiments, otoscope 300 has a curved shape. For example, otoscope 300 has a handle portion in which one or more electronic circuitry 308 is located. In some embodiment, the handle portion of otoscope 300 is ergonomically shaped for a user to hold otoscope 300. In some embodiments, otoscope 300 has a probe portion. In some embodiments, the probe portion of otoscope 300 is orthogonal to the handle portion (e.g., the curve is a 90 degree angle). In some embodiments, the probe portion includes an otoscope tip 306 and a speculum 304.

In some embodiments, as described above, speculum 304 is attached to otoscope 300 through any number of the methods described above. In some embodiments, the housing of otoscope 300 includes an attachment tip 306. In some embodiments, attachment tip 306 extends outwards and forwards from otoscope 300. In some embodiments, speculum 304 is a rubber, polymer, or otherwise flexible material. In some embodiments, speculum 304 is attached to otoscope tip 306 via the natural clamping force of the rubber, polymer, or other flexible material of speculum 304. For example, to attach speculum 304 to otoscope tip 306, a user pushes speculum 304 onto otoscope tip 306, causing a portion of speculum 304 to be displaced by the otoscope tip 306. In some embodiments, due to the elastic nature of speculum 304, the speculum 304 is clamped onto otoscope tip 306, thus causing a temporary attachment between otoscope 300 and speculum 304. In some embodiments, the temporary attachment allows for quick disposal of used speculums and installation of a fresh, sterile speculum, thus minimizing cross-contamination between uses and/or users.

In some embodiments, otoscope 300 includes a camera 310. In some embodiments, camera 310 is placed inside of otoscope tip 306. In some embodiments, a mounting mechanism attaches to camera 310 and secures camera 310 inside of otoscope tip 306. In some embodiments, the outer plane of camera 310 is flush with the outer plane of otoscope tip 306. In some embodiments, camera 310 includes one or more lighting elements are aligned along the circular edge (e.g., circumference) of camera 310. In some embodiments, placing camera 310 as close to the interrogated area of the patient (e.g., ear canal) increases the resolution and quality of the captured images. In some embodiments, camera 310 can be placed anywhere within otoscope 300 and one or more lenses can direct light into camera 310. In some embodiments, one or more electronics for driving and/or controlling camera 310 are attached to camera 310. In some embodiments, the one or more electronics are integrated into the mounting mechanism and/or perform the function of providing mechanical stability in maintaining camera 310 at the appropriate position inside otoscope tip 306.

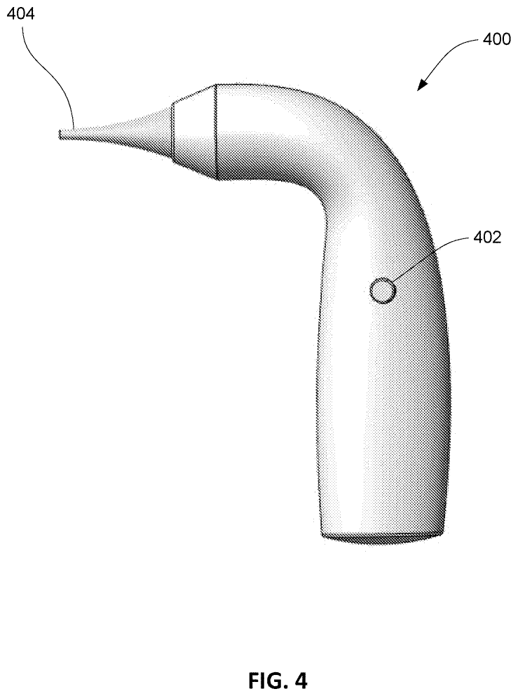

In some embodiments, images taken by camera 310 are sent (e.g., by wire) to electronics 308. In some embodiments, electronics 308 includes one or more of communication circuitry (e.g., wireless or wired), processing circuitry, camera control circuitry, battery circuitry and/or batteries. In some embodiments, these circuits are similar to those described above with respect to FIGS. 1-2. In some embodiments, otoscope 300 includes a physical button (e.g., button 402 as described below with respect to FIG. 4) on the outer body of otoscope 300. In some embodiments, the physical button is selectable to cause camera 310 to capture an image. In some embodiments, when camera 310 captures an image, the image is transmitted to a host device (e.g., similar to the method described above with respect to FIGS. 1-2.) In some embodiments, otoscope 300 performs one or more initial processing steps before transmitting the images to the host device. In some embodiments, otoscope 300 stores one or more images temporary on an onboard memory (e.g., RAM) before transmitting the images to the host device.

In some embodiments, the physical button is selectable to cause otoscope 300 to power on. In some embodiments, when the physical button is selectable to cause otoscope 300 to power on, camera 310 is controlled by a signal from the host device. For example, while otoscope 300 and the host device have an active communication connection (e.g., via Bluetooth, RF, IR, NFC, WiFi, WiFi Direct, etc.), host device can transmit a camera capture command to otoscope 300. In some embodiments, in response to the capture command, otoscope 300 causes camera 310 to capture an image. In some embodiments, after capturing the image, otoscope 300 transmits the image to the host device.

In some embodiments, otoscope 300 includes a charging port 312. In some embodiments, charging port 312 is a Universal Serial Bus (USB) port which is configured to accept power through the port 312. In some embodiments, the power received at port 312 charges the onboard batteries in otoscope 300. In some embodiments, the batteries in otoscope 300 are rechargeable, replaceable, and/or removable. In some embodiments, port 312 also supports wired communication with a host device. For example, a USB cable can be connected from port 312 to a host device. In some embodiments, when a USB cable is used as a communication medium, the communication circuitry in otoscope 300 disables the wireless mode and transmits the captured images over the wired communication medium.

FIG. 4 illustrates an exterior view of an exemplary otoscope 400. In some embodiments, otoscope 400 is similar to or shares features of otoscope 300. In some embodiments, otoscope 400 has a curved shape which includes a handle portion and a probe portion. In some embodiments, the probe portion of otoscope 400 attaches to speculum 404. In some embodiments, as described above with respect to FIG. 3, the handle portion of otoscope 400 includes button 402. In some embodiments, button 402 can be a physical button (e.g., mechanical button) or a capacitive touch button. It is understood that button 402 can be placed anywhere on the body of otoscope 400 and is not limited to the location shown in FIG. 4.

In some embodiments, button 402 is selectable to cause otoscope 400 to power on and/or power-off. For example, if otoscope 400 is currently on, button 402 is selectable to cause otoscope 400 to power off and if otoscope 400 is currently off, button 402 is selectable to cause otoscope 400 to power on. In some embodiments, a light or other suitable indicator indicates to the user that otoscope 400 is powered on. In some embodiments, when otoscope 400 is powered on, otoscope 400 connects to the host device and the host device indicates to the user that otoscope 400 is powered on (e.g., by displaying images captured by the camera in otoscope 400, displaying a notification, or displaying any other suitable indicator that otoscope 400 is powered on and connected to the host device).

In some embodiments, button 402 is selectable to cause the camera to capture an image and send the image to the host device. In some embodiments, the camera only captures images when the user actuates button 402. In some embodiments, the camera continuously captures images and only transfers the images to the host device upon actuation of button 402. In some embodiments, the camera continuously captures images and transfers the images to the host device and actuation of button 402 causes transmission of a signal to capture (e.g., save) the image at the time of button 402 actuation. In some embodiments, otoscope 400 is not continuously capturing and/or transmitting images to the host device and in response to the host device receiving a user input to capture the image, the host device transmits a signal (e.g., a command) to otoscope 400 to capture and/or transmit an image to the host device. In some embodiments, the host device is continuously receiving images from otoscope 400 and receiving a user input to capture the image causes the host device to save the image at the time when the user input is received.

Therefore, according to the above, some embodiments of the disclosure are directed to an apparatus. In some embodiments, the apparatus comprises a housing; a light source disposed within the housing; an speculum attached to the housing, the speculum configured to pass light from the light source through a first opening of the speculum and through a second opening of the speculum; a plurality of image sensing elements disposed within the housing and configured to sense an image via light received through the speculum; electronic circuitry configured to process the sensed image; and communication circuitry electrically coupled to the plurality of image sensing elements, the communication circuitry configured to transmit the sensed image to a hand-held mobile device.

Additionally or alternatively, the communication circuitry is configured to transmit the sensed image to the host processor wirelessly. Additionally or alternatively, the optical redirection device is disposed between the speculum and the plurality of image sensing elements. Additionally or alternatively, the apparatus further comprises a flexible optical guide configured to direct light from the light source through the speculum. Additionally or alternatively, the apparatus further comprises an optical redirection component configured to direct returned light to the plurality of image sensing elements, the returned light passing through the second opening and through the first opening. Additionally or alternatively, the light source is disposed around the plurality of image sensing elements.

Some embodiments of the disclosure are directed to a method. In some embodiments, the method comprises: at an otoscope having a light source, an speculum, a plurality of image sensing elements, and a communication circuitry electrically coupled to the plurality of image sensing elements: transmitting light from the light source through the speculum, the light first passing through a first opening of the speculum and through a second opening of the speculum; sensing, using the plurality of image sensing elements, an image through the speculum; processing, using the electronic circuitry, the image into transmittable data format; and transmitting, using the communication circuitry, the image to a hand-held mobile device.

Additionally or alternatively, the communication circuitry transmits the sensed image wirelessly to the hand-held mobile device. Additionally or alternatively, the method further comprises directing light from the light source through the speculum using a flexible optical guide disposed in the housing. Additionally or alternatively, the method further comprises directing, using an optical redirection device, returned light to the plurality of image sensing elements, the returned light passing through the second opening and through the first opening. Additionally or alternatively, the method further comprises analyzing the image to determine an existence of one or more physiological characteristics. Additionally or alternatively, the one or more physiological characteristics include at least one of an ear infection or fluid buildup. Additionally or alternatively, analyzing the image to determine the existence of the one or more physiological characteristics is performed using a machine learning module. Additionally or alternatively, the method further comprises training the machine learning model using a plurality of images that have been identified as including the one or more physiological characteristics

Although the disclosed examples have been fully described with reference to the accompanying drawings, it is to be noted that various changes and modifications will become apparent to those skilled in the art. Such changes and modifications are to be understood as being included within the scope of the disclosed examples as defined by the appended claims.

1. An apparatus, comprising:
a housing;
a light source disposed within the housing;
an speculum attached to the housing, the speculum configured to pass light from the light source through a first opening of the speculum and through a second opening of the speculum;
a plurality of image sensing elements disposed within the housing and configured to sense an image via light received through the speculum;
electronic circuitry configured to process the sensed image; and
communication circuitry electrically coupled to the plurality of image sensing elements, the communication circuitry configured to transmit the sensed image to a hand-held mobile device.

2. The apparatus of claim 1, wherein the communication circuitry is configured to transmit the sensed image to the host processor wirelessly.

3. The apparatus of claim 1, wherein the optical redirection device is disposed between the speculum and the plurality of image sensing elements.

4. The apparatus of claim 1, further comprising a flexible optical guide configured to direct light from the light source through the speculum.

5. The apparatus of claim 1, further comprising an optical redirection component configured to direct returned light to the plurality of image sensing elements, the returned light passing through the second opening and through the first opening.

6. The apparatus of claim 1, wherein the light source is disposed around the plurality of image sensing elements.

7. A method, comprising:
at an otoscope having a light source, a speculum, a plurality of image sensing elements, and a communication circuitry electrically coupled to the plurality of image sensing elements:
transmitting light from the light source through the speculum, the light first passing through a first opening of the speculum and through a second opening of the speculum;
sensing, using the plurality of image sensing elements, an image through the speculum;
processing, using the electronic circuitry, the image into transmittable data format; and
transmitting, using the communication circuitry, the image to a hand-held mobile device.

8. The method of claim 7, wherein the communication circuitry transmits the sensed image wirelessly to the hand-held mobile device.

9. The method of claim 7, further comprising:
directing light from the light source through the speculum using a flexible optical guide disposed in the housing.

10. The method of claim 7, further comprising:
directing, using an optical redirection device, returned light to the plurality of image sensing elements, the returned light passing through the second opening and through the first opening.

11. The method of claim 7, further comprising:
analyzing the image to determine an existence of one or more physiological characteristics.

12. The method of claim 11, wherein the one or more physiological characteristics include at least one of an ear infection or fluid buildup.

13. The method of claim 11, wherein analyzing the image to determine the existence of the one or more physiological characteristics is performed using a machine learning module.

14. The method of claim 13, further comprising:
training the machine learning model using a plurality of images that have been identified as including the one or more physiological characteristics.
